(19) United States
(12) Patent Application Publication
(10) Pub. No.: US 2016/0263258 A1
(43) Pub. Date: Sep. 15, 2016

(54) Radiotracer introduced [18F]fluoromethyl group targeting neuroinflammation for PET imaging and Synthesis of Radiotracer and its biological evaluation Method for Radiotracer

(71) Applicant: Bio Imaging Korea Co., Ltd., Seoul (KR)

(72) Inventors: Byung Chul Lee, Seoul (KR); Byung Seok Moon, Seongnam (KR); Jae Ho Jung, Seoul (KR)

(73) Assignee: Bio Imaging Korea Co., Ltd.

(21) Appl. No.: 15/069,403

(22) Filed: Mar. 14, 2016

Related U.S. Application Data
(63) Continuation of application No. PCT/KR2013/009387, filed on Oct. 21, 2013.

(30) Foreign Application Priority Data
Sep. 13, 2013 (KR) 10-2013-0110282

(51) Int. Cl. A61K 51/04; C07B 59/00; C07D 401/04

(52) U.S. Cl. CPC A61K 51/0455; C07D 401/04; C07B 59/002; C07B 2200/05

(57) ABSTRACT

Disclosed are an [¹⁸F]fluoromethyl group-introduced radiotracer for brain neuroinflammation-targeting positron emission tomography (PET), the synthesis thereof, and a method for evaluating biological results using the same. In the method for the synthesis of an [¹⁸F]fluoromethyl group-introduced radiotracer for brain neuroinflammation-targeting positron emission tomography, a compound obtained by introducing triazolium triflate into normethyl-PBR28 is used as a precursor and a fluoromethyl group is labeled with fluorine-18 in a single step. The [¹⁸F]fluoromethyl group-introduced radiotracer for brain neuroinflammation-targeting positron emission tomography is synthesized by using a compound, obtained by introducing triazolium triflate into normethyl-PBR28, as a precursor and performing substitution with fluorine-18 in a single step.

### CROSS-REFERENCE TO RELATED APPLICATION

This application is a continuation of PCT/KR2013/009387 filed on Oct. 21, 2013, which claims priority to Korean Application No. 10-2013-0110282 filed on Sep. 13, 2013, which applications are incorporated herein by reference.

### TECHNICAL FIELD

The present invention relates generally to an [¹⁸F]fluoromethyl group-introduced radiotracer for brain neuroinflammation-targeting positron emission tomography, the synthesis thereof, and a method for evaluating biological results using the same, and more particularly to N-(2-[¹⁸F]fluoromethoxybenzyl)-N-(4-phenoxypyridin-3-yl)acetamide which can evaluate usefulness for the imaging of brain neuroinflammation via positron emission tomography (PET) using a radiotracer for the selective PET imaging of peripheral benzodiazepine receptor (PBR), the synthesis thereof, and the evaluation of in vitro binding affinity, lipophilicity, and pharmacokinetics in brain neuroinflammation models using the same.

### BACKGROUND ART

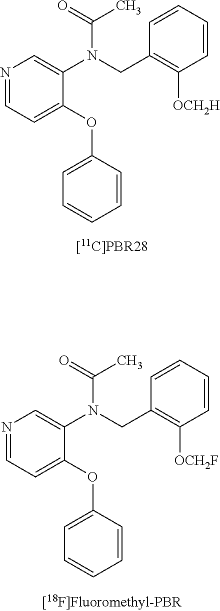

The microglial cells of the central nervous system contribute to the activation and maintenance of homeostasis of the nervous system, and function to secrete neurotrophins, nitric oxide, inflammation-causing cytokines or the like to thereby maintain neurons or cause the apoptosis of neurons. In fact, the activation of microglial cells in diseases, including various neurodegenerative diseases such as Alzheimer's disease, Parkinson's disease and Huntington's disease, cerebral infarction or injury and brain infection, has been reported. In addition, it is known that the deposition of beta-amyloid, which contributes to the development and progression of Alzheimer's disease, induces the activation of microglial cells.

Recently, it has been reported that the activation of microglial cells is caused by an increase in the expression of mitochondrial 18-kDa translocator protein (TSPO), and begins within a few hours after the onset of a disease and then continues for a few days. Accordingly, the measurement of the expression level of TSPO in microglial cells in various diseases of the central nervous system can be used as an in vivo biomarker that evaluates cell activation during neuroinflammatory processes. In fact, [C]-(R)-PK11195 ((R)-N-methyl-N-(1-methylpropyl)-1-(2-chlorophenyl)isoquinoline-3-carboxamide) labeled with C-11 (half-life: 20.4 minutes) as a positron emission tomography (PET) radiotracer for the evaluation of TSPO expression was first developed in 1984, and is known to bind to isoquinoline binding protein (IBP).

However, the wide use of [¹¹C]-(R)-PK11195 has been limited due to problems such as the short half-life of the radioisotope C-11 and the non-specific binding and low signal-to-noise ratio of the ligand PK11195. As a result, a variety of new radiotracers for imaging of brain neuroinflammation have been developed over the course of the past twenty years, and typical examples thereof include [¹¹C]DAA1106 (N-5-fluoro-2-phenoxyphenyl)-N-(2,5-dimethoxybenzyl)acetamide) whose uptake is at least 4 times or higher than that of [¹¹C]-(R)-PK11195 and whose metabolites do not pass through the blood brain barrier. However, it was reported that [¹¹C]DAA1106 also has the problem of showing a low specific signal for TSPO. [¹¹C]PBR28 (N-acetyl-N-(2-[¹¹C]methoxybenzyl)-2-phenoxy-5-pyridinamine) was developed to overcome the pharmacokinetic disadvantages of [¹¹C]DAA1106 shows a high signal-to-noise ratio while maintaining the basic chemical structure of [¹¹C]DAA1106, indicating that its effectiveness as a radiotracer for imaging of brain neuroinflammation was verified. Thus, clinical studies on [¹¹C]PBR28 have been conducted. However, because [¹¹C]PBR28 is also a compound labeled with carbon-11 having a short half-life, this radiotracer has disadvantages in that it can be used only for a short time after its production, is highly likely to cause radiation poisoning, and can be applied only to two or less patients depending on the number of held positron emission tomography (PET) systems after it is produced by one production process.

In contrast, fluorine-18, which is another positron-emitting radionuclide, has a relatively long half-life (t_{1/2}=109.8 minutes), and can be applied to diagnosis using radiotracers in a plurality of PET systems over a long period of time after production, because a method of labeling a target compound with fluorine-18 by organic synthesis is easily performed.

Therefore, there is a need for a radiotracer which can be conveniently and efficiently labeled with the radioisotope fluorine-18 and, at the same time, can selectively target brain neuroinflammation. However, a change in the structure of [¹¹C]PBR28 whose superiority has been proven is necessarily required to introduce fluorine-18 thereto, and this structural change results in a change in the biological properties of [¹¹C]PBR28.

In order to label [¹¹C]PBR28 with fluorine-18 while minimizing the structural change of [¹¹C]PBR28, the present inventors have designed a novel structure which has a fluoromethyl group introduced thereto and which is obtained by substituting a hydrogen atom in the original structure of [¹¹C]PBR28 with a fluorine atom, and have determined that the novel structure can overcome the above-described disadvantages, thereby completing the present invention.

A pharmacologically active compound containing a fluoromethyl group having the same structure as a carbon-11-labeled methoxy group has a molecular formula of R-CH₂F (where R is a drug) different from the molecular formula (R-CH₂H) of the compound containing the methoxy group, and is a compound obtained by substituting the hydrogen (H) atom of R-CH₂H with a fluorine (F) atom. These compounds are structurally similar in that the van der Waals radii from the adjacent carbon atom are 1.20 Å (H) and 1.47 Å (F). Studies on the application of various active drugs reported that, when the hydrogen atom of the active drug was substituted with a fluorine atom, the target binding affinity of the active drug was increased and the efficiency of passage through the blood-brain barrier (BBB) was increased in the case of central nervous system drugs.

In a method for introducing an [¹⁸F]fluoromethyl group, the phenol position of a target active drug can be selectively labeled with fluorine-18 either through a two-step reaction using a prosthetic group or through a single-step reaction after the introduction of a triazolium triflate leaving group. Thus, the diagnosis of neurodegenerative disease using a fluorine-18-labeled radiotracer for targeted imaging of brain neuroinflammation is required, and for this purpose, the synthesis of an [¹⁸F]fluoromethyl-peripheral benzodiazepine receptor radiotracer ([¹⁸F]fluoromethyl-PBR radiotracer) and the evaluation of the usefulness thereof are required.

Technology related to the in vivo imaging of PBR as described above is disclosed in Korean Unexamined Patent Application Publication No. 2011-0071072.

Hereinafter, a method for the imaging of neuroinflammation disclosed in Korean Unexamined Patent Application Publication No. 2011-0071072 is briefly described as conventional technology.

FIG. 1 is a graph showing the relative intensity of in vivo imaging agent 1 binding in the facial nucleus of a rat seven days post-FNA in Korean Unexamined Patent Application Publication No. 2011-0071072 (hereinafter referred to as “conventional technology”). As shown in FIG. 1, the conventional method for imaging of neuroinflammation includes: (i) administering to a subject an in vivo imaging agent as defined in any one of claims 1 to 16; (ii) allowing the in vivo imaging agent to bind to PBR in the subject; (iii) detecting by an in vivo imaging procedure signals emitted by the radioisotope of the in vivo imaging agent; (iv) generating an image representative of the location and/or amount of the signals; and (v) determining the distribution and extent of PBR expression in the subject, wherein the expression is directly correlated with the signals emitted by the in vivo imaging agent.

However, the method for the imaging of neuroinflammation according to the conventional technology is problematic in that it is difficult to evaluate the usefulness of a radioactive material. Therefore, there is a demand for a method for evaluating the usefulness of a radioactive material.

### SUMMARY OF THE DISCLOSURE

An object of the present invention is to overcome the above-described problems of the conventional technology. The present inventors have synthesized an [¹⁸F]fluoromethyl group-introduced radiotracer as a novel neuroinflammation-targeting PET radiotracer, have evaluated binding affinity, lipophilicity, and pharmacokinetics in neuroinflammatory models, and, as a result, have found that the [¹⁸F]fluoromethyl group-introduced radiotracer provides an image superior to that of an existing carbon-11-labeled brain neuroinflammation-targeting radiotracer, thereby completing the present invention.

The present inventors have developed a fluorine-18-labeled radiotracer which is synthesized with high radiochemical yield and high specific activity within a short process time by use of the above-described method of introducing an [¹⁸F]fluoromethyl group using a prosthetic group or a triazolium triflate precursor, and have verified that the usefulness of the fluorine-18-labeled radiotracer for selective PET imaging of brain neuroinflammation, thereby completing the present invention.

Therefore, an object of the present invention is to provide an [¹⁸F]fluoromethyl group-introduced radiotracer for brain neuroinflammation-targeting positron emission tomography which is synthesized using the positron emission radionuclide fluorine-18 radioisotope that is highly applicable to the diagnosis of brain neuroinflammation diseases and which has a high affinity for a peripheral benzodiazepine receptor and also can provide ideal pharmacokinetic information for the imaging of brain neuroinflammation, the synthesis thereof, and a method for evaluating biological results using the same.

In order to achieve the above-described objects, the present invention provides a method for the synthesis of an [¹⁸F]fluoromethyl group-introduced radiotracer for brain neuroinflammation-targeting positron emission tomography, wherein a compound obtained by introducing triazolium triflate into normethyl-PBR28 is used as a precursor and a fluoromethyl group is labeled with fluorine-18 in a single step.

In the present invention, a reference material for the [¹⁸F]fluoromethyl group-introduced radiotracer may be N-(2-fluoromethoxybenzyl)-N-(4-phenoxypyridin-3-yl)acetamide which is synthesized either by introducing [¹⁹F]fluoroiodomethane using normethyl-PBR28 as a starting material or by subjecting a triazolium triflate precursor to a substitution reaction with fluorine-19 using tetrabutylammonium fluoride (TBAF), and which is used to identify the [¹⁸F]fluoromethyl group-introduced radiotracer through the simultaneous injection of the reference material and the [¹⁸F]fluoromethyl group-introduced radiotracer into HPLC and is also used to evaluate the binding affinity of the [¹⁸F]fluoromethyl group-introduced radiotracer for TSPO.

In the present invention, 1-(chloromethyl)-3-methyl-4-phenyl-1H-1,2,3-triazol-3-ium triflate obtained using 1-(chloromethyl)-4-phenyl-1H-1,2,3-triazole and MeOTf may be used as an intermediate for the synthesis of the precursor labeled with fluorine-18.

The present invention also provides a method for evaluating biological results using an [¹⁸F]fluoromethyl group-introduced radiotracer for brain neuroinflammation-targeting positron emission tomography, which is synthesized by using a compound, obtained by introducing triazolium triflate into normethyl-PBR28, as a precursor and performing substitution with fluorine-18 in a single step, the method including: by using the [¹⁸F]fluoromethyl group-introduced radiotracer, evaluating the specificity of the [¹⁸F]fluoromethyl group-introduced radiotracer using PK11195 (8-12 mg/kg) and fluoromethyl-PBR28 (3-7 mg/kg) which are standard materials, and evaluating the selectivity of the [¹⁸F]fluoromethyl group-introduced radiotracer using flumazenil (3-7 mg/kg) which binds to central benzodiazepine receptor (CBR).

The present invention also provides an [¹⁸F]fluoromethyl group-introduced radiotracer for brain neuroinflammation-targeting positron emission tomography, which is synthesized by using a compound, obtained by introducing triazolium triflate into normethyl-PBR28, as a precursor and performing substitution with fluorine-18 in a single step.

### BRIEF DESCRIPTION OF THE DRAWINGS

FIG. 1 is a graph showing the relative intensity of in vivo imaging agent 1 binding in the facial nucleus of a rat seven days post-FNA according to a conventional technology;

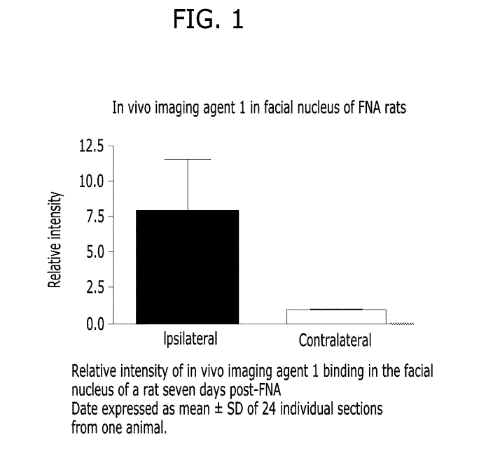

FIG. 2 shows the structural formulas of [¹¹C]PBR28 and an [¹⁸F]fluoromethyl group-introduced radiotracer ([¹⁸F]fluoromethyl-PBR);

FIG. 3 shows reaction schemes showing a method for the synthesis of an [¹⁸F]fluoromethyl group-introduced radiotracer;

FIG. 4 is an HPLC chromatogram showing the results of separating a pure [¹⁸F]fluoromethyl group-introduced radiotracer from a synthetic mixture in order to evaluate usefulness for brain neuroinflammation;

FIG. 5 is an HPLC chromatogram showing the results of an HPLC experiment in which an [¹⁸F]fluoromethyl group-introduced radiotracer prepared in order to evaluate usefulness for brain neuroinflammation was injected into an HPLC simultaneously with a reference material having a non-radioisotope in order to check whether the [¹⁸F]fluoromethyl group-introduced radiotracer is the same as a reference material having a non radioisotope;

FIG. 6 is a graph showing the time-dependent uptake and discharge of [¹¹C]PBR28 and an [¹⁸F]fluoromethyl group-introduced radiotracer between a brain inflammation-induced portion and a normal brain portion in the same brain neuroinflammation model in an experiment performed to evaluate usefulness for brain neuroinflammation;

FIG. 7 shows positron emission tomography images acquired after injecting an [¹⁸F]fluoromethyl group-introduced radiotracer simultaneously with PK11195, a fluorine-19-labeled reference material and flumazenil in order to evaluate the selectivity and specificity of the [¹⁸F]fluoromethyl group-introduced radiotracer in the evaluation of usefulness for brain neuroinflammation; and

FIG. 8 shows HPLC chromatograms showing the results of measuring the metabolism of an [¹⁸F]fluoromethyl group-introduced radiotracer in a brain extracted after the intravenous injection of the [¹⁸F]fluoromethyl group-introduced radiotracer in order to evaluate usefulness for brain neuroinflammation.

### DETAILED DESCRIPTION OF THE DISCLOSURE

The terms and words used in the specification and the claims should be interpreted as having meanings and concepts relevant to the technical scope of the present invention, based on the principle according to which the inventors can appropriately define the concept of the terms in order to describe their invention in the best manner.

Throughout the specification and the claims, when any component “includes (or comprises)” any component, this is not intended to exclude other components, but may further include other components, unless otherwise specified.

Hereinafter, embodiments of an [¹⁸F]fluoromethyl group-introduced radiotracer for brain neuroinflammation-targeting positron emission tomography, the synthesis thereof and a method for evaluating biological results using the same according to the present invention are described in detail with reference to the accompanying drawings.

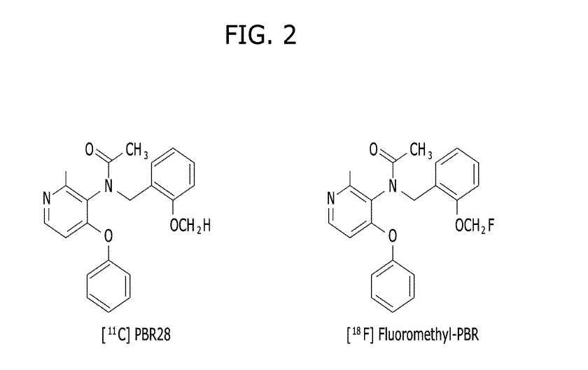

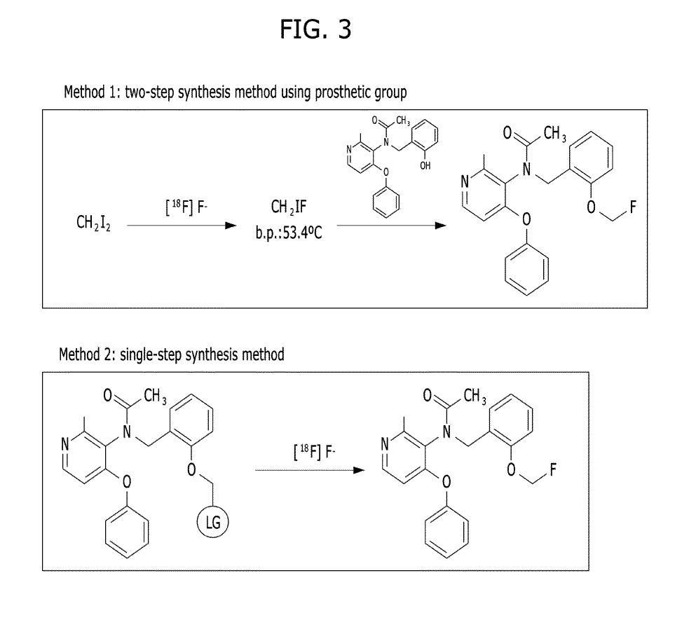

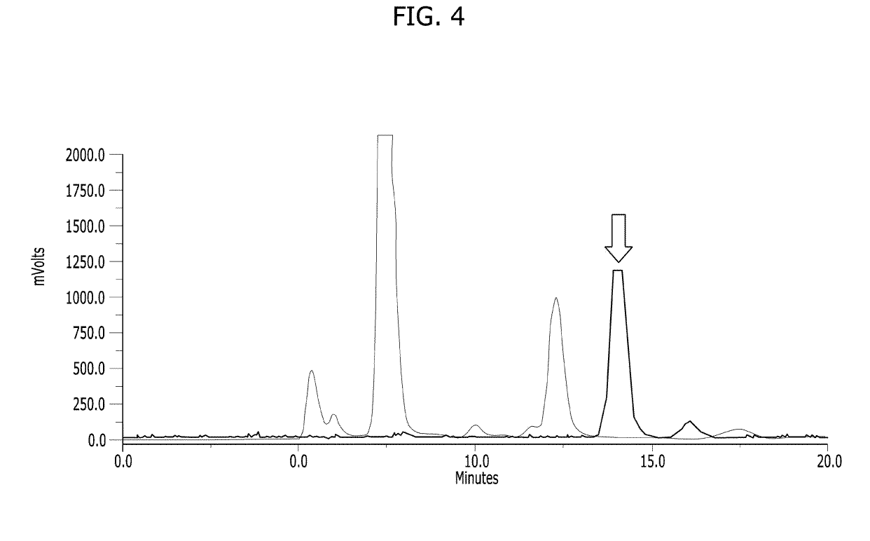

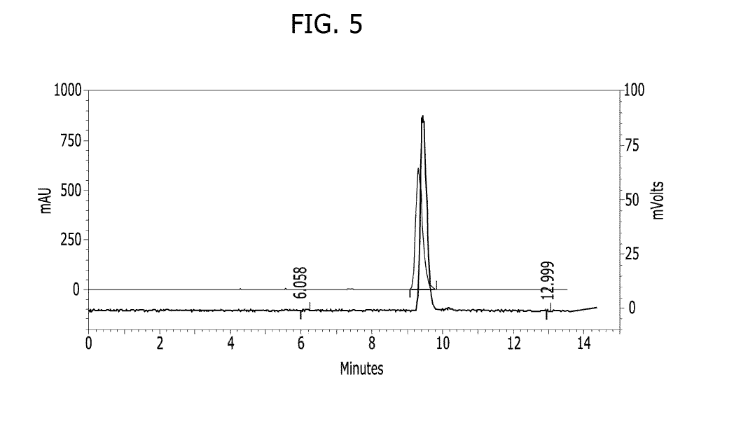

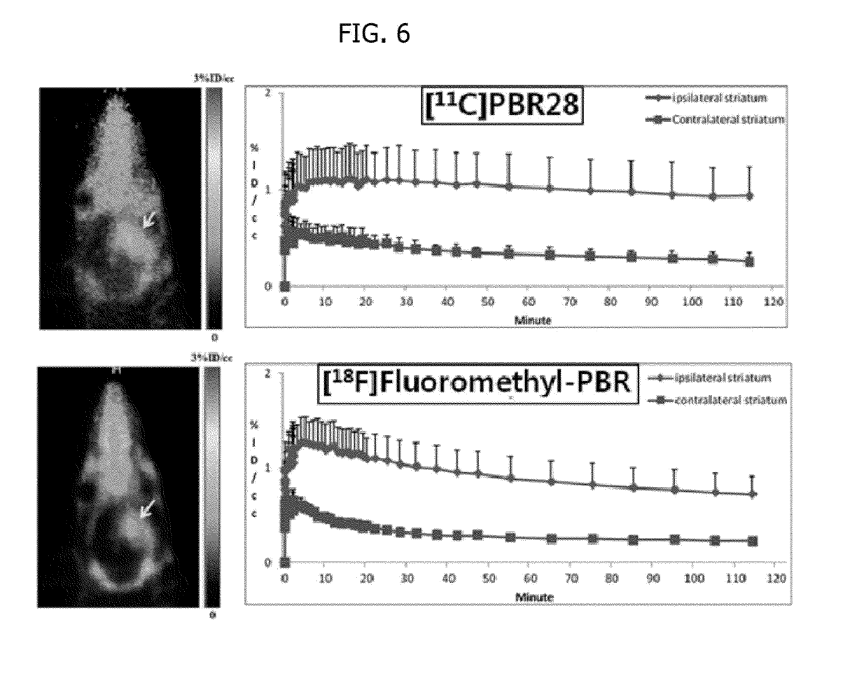

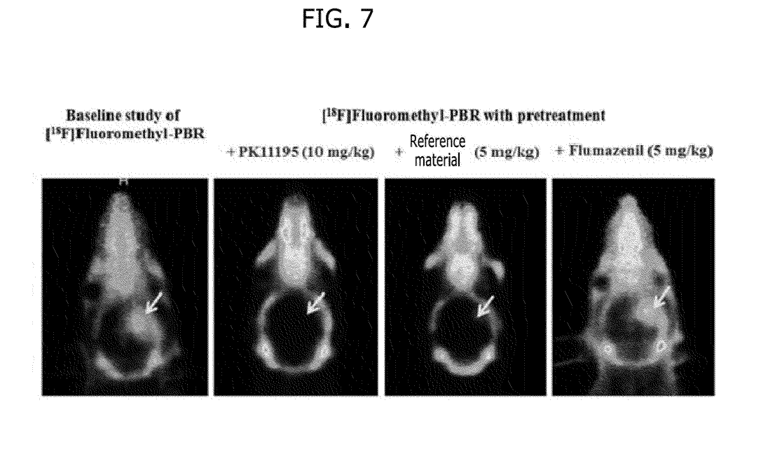

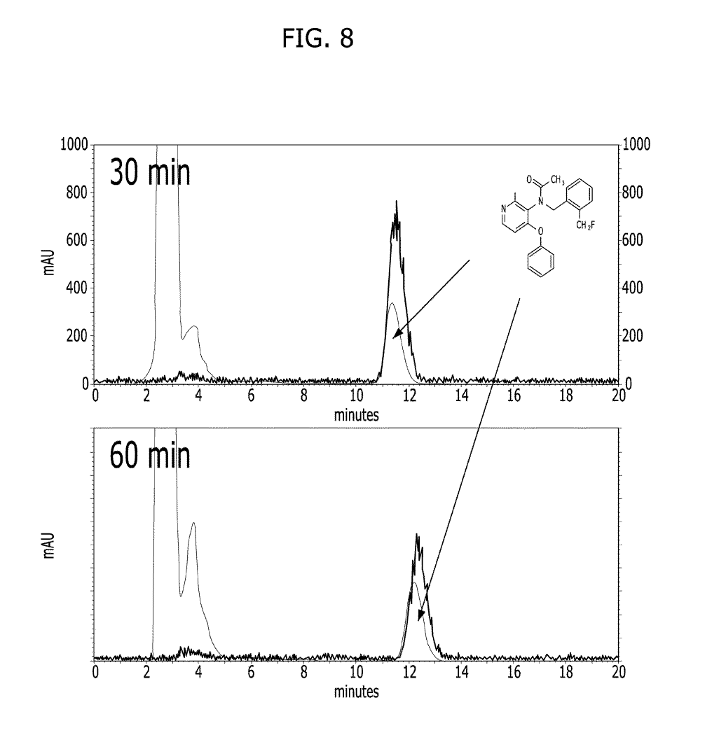

FIG. 2 shows the structural formulas of [¹¹C]PBR28 and an [¹⁸F]fluoromethyl group-introduced radiotracer; FIG. 3 shows reaction schemes showing a method for the synthesis of an [¹⁸F]fluoromethyl group-introduced radiotracer; FIG. 4 is an HPLC chromatogram showing the results of separating a pure [¹⁸F]fluoromethyl group-introduced radiotracer from a synthetic mixture in order to evaluate the usefulness of the radiotracer for imaging of brain neuroinflammation; FIG. 5 is an HPLC chromatogram showing the results of an HPLC experiment in which an [¹⁸F]fluoromethyl group-introduced radiotracer prepared in order to evaluate usefulness for brain neuroinflammation was injected into an HPLC simultaneously with a reference material having a non-radioisotope in order to check whether the [¹⁸F]fluoromethyl group-introduced radiotracer is the same as a reference material having a non radioisotope; FIG. 6 is a graph showing the time-dependent uptake and discharge of [¹¹C]PBR28 and an [¹⁸F]fluoromethyl group-introduced radiotracer between a brain inflammation-induced portion and a normal brain portion in the same brain neuroinflammation model in an experiment performed to evaluate usefulness for brain neuroinflammation; FIG. 7 shows positron emission tomography images acquired after injecting an [¹⁸]fluoromethyl group-introduced radiotracer simultaneously with PK11195, a fluorine-19-labeled reference material and flumazenil in order to evaluate the selectivity and specificity of the [¹⁸F]fluoromethyl group-introduced radiotracer in the evaluation of usefulness for brain neuroinflammation; and FIG. 8 shows HPLC chromatograms showing the results of measuring the metabolism of an [¹⁸F]fluoromethyl group-introduced radiotracer in a brain extracted after the intravenous injection of the [¹⁸F]fluoromethyl group-introduced radiotracer in order to evaluate usefulness for brain neuroinflammation.

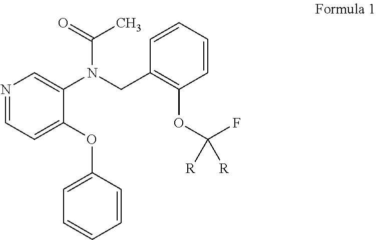

where R is H or deuterium (D).

In a method for the production of an [¹⁸F]fluoromethyl group-introduced radiotracer according to an embodiment of the present invention, labeling with fluorine-18 may be performed according to two methods by use of a prosthetic group or a precursor, as shown in reaction schemes 1 and 2 below. As used herein, the term “[¹⁸F]fluoromethyl group-introduced radiotracer” refers to an ¹⁸F-labeled fluoromethyl ether derivative that is a novel brain neuroinflammation-targeting PET radiotracer. The term “PBR” refers to a peripheral type benzodiazepine receptor.

First, methods for the synthesis of a prosthetic group and a precursor, which are used for the labeling of a fluoromethyl group with fluorine-18, are described with reference to reaction schemes 1 and 2 below.

Reaction Scheme 1: Two-Step Preparation Method for Fluorine-18 Labeling Using Prosthetic Group

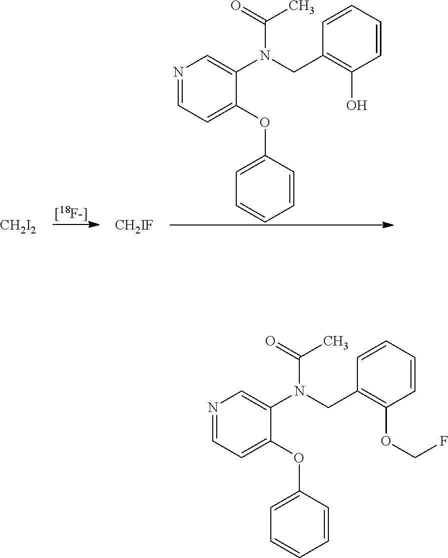

First, a fluorine-18 substitution reaction is performed from diiodomethane purchasable from a reagent company to prepare iodo[¹⁸F]fluoromethane which is then purified using a Sep-Pak cartridge, followed by alkylation with normethyl-PBR28, thereby preparing the final target compound.

Reaction Scheme 2: Single-Step Preparation Method for Fluorine-18 Labeling

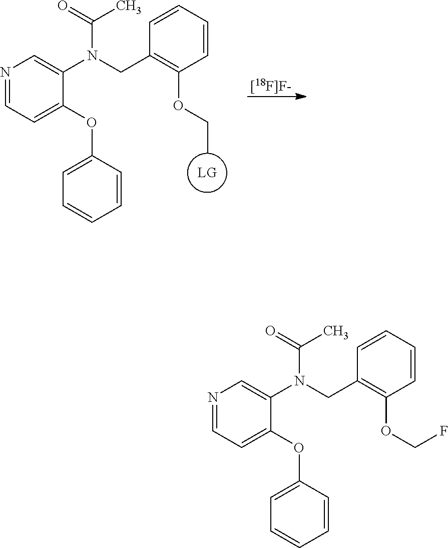

In the single-step preparation method for fluorine-18 labeling, a precursor is prepared by introducing a suitable leaving group (LG) into normethyl-PBR28, and then labeled with fluorine-18, thereby the final target compound. In this case, 1-(chloromethyl)-3-methyl-4-phenyl-1H-1,2,3-triazol-3-ium triflate may be used as the leaving group.

A fluorine-19-substituted reference material is synthesized by performing a substitution reaction on normethyl-PBR28 using tetrabuthylammonium fluoride substituted with fluorine-19 instead of fluorine 18.

In the two-step labeling method using a prosthetic group, fluorine-18 produced in a cyclotron is adsorbed on the Chromafix® (PS-HCO₃) cartridge, followed by elution with methanol/water containing a phase transfer catalyst. The resulting eluate is dried by azeotropic distillation, and diiodomethane is added thereto. The reaction mixture is heated at 90° C. for about 15 minutes and purified by a Silica Sep-Pak cartridge. The purified iodo[¹⁸F]fluoromethane is subjected to an alkylation reaction with normethyl-PBR28 at 90° C. for about 5 minutes, followed by purification using an HPLC system. To remove the clinically unacceptable HPLC solvents, the collected solution is prepared into a 5% ethanol/saline solution using a tC18 Sep-Pak cartridge.

Reaction conditions for the single-step labeling method using the triazolium triflate precursor are as follows.

Fluorine-18 produced in a cyclotron is adsorbed on the Chromafix® (PS-HCO₃) cartridge, followed by elution with methanol/water containing a phase transfer catalyst. The resulting eluate is dried by azeotropic distillation, and a triazolium triflate precursor is added thereto. The reaction mixture is heated at 120° C. for 10 minutes, and then cooled to room temperature, followed by purification using a Sep-Pak cartridge. The eluted solution is purified using an HPLC system. To remove the clinically unacceptable HPLC solvents, the collected solution is prepared into a 5% ethanol/saline solution using a tC18 Sep-Pak cartridge.

In the following examples, reagents and solvents purchasable from reagent companies were used without purification, except for special cases. The reagents and solvents used were purchased from Sigma-Aldrich (in the U.S.A.). In each reaction, chromatography for purification was performed using silica gel (Merck, 230-400 mesh, ASTM), and all reactions were observed using a pre-coated plate (Merck, silica gel 60F₂₅₄). ¹H and ¹³C NMR spectra were analyzed with Varian 500-MR (500 MHz) spectrometer, and expressed as parts per million (ppm, d units). Water (H₂ ¹⁸O) used was purchased from Taiyo Nippon Sanso Corporation (in Japan), and fluorine-18 was produced at Seoul National University Bundang Hospital (in Korea) by an ¹⁸O(p,n)¹⁸F reaction through proton irradiation using a KOTRON-13 cyclotron (from Samyoung Unitech Co., Ltd.). Chromafix®-HCO₃ (45 mg) cartridges were purchased from Macherey-Nagel Ins. (Germany), and C18 plus Sep-Pak®8 cartridges were purchased from Waters Corp. (in the U.S.A.). HPLC was performed using a Gilson 322 column equipped with a NaI radiodector (Raytest) and a UV detector, and HPLC-grade solvents (J. T. Baker, U.S.A.) were used for HPLC purification after membrane filtration (Whatman, 0.22 μm). Radio-TLC was analyzed on a Bioscan radio-TLC scanner (from Washington DC, USA). All radioactivities were measured using a VDC-505 activity calibrator from Veenstra Instruments (in Netherlands), and radiochemical yields were expressed after decay-correction.

### Example 1

Hereinafter, a method of preparing the final target compound using the two-step fluorine-18 labeling method is described.

Fluorine-18 produced in a cyclotron was absorbed onto the Chromafix® (PS-HCO₃) cartridge, followed by elution with methanol/water containing a phase transfer catalyst such as tetrabutylammonium bicarbonate. The resulting eluate was dried by azeotropic distillation, and a solution of diiodomethane (50 μL) in acetonitrile (0.4 mL) was added thereto. The reaction mixture was heated at 90° C. for 15 minutes, passed through a Silica Sep-Pak cartridge, and collected in DMF. Normethyl-PBR28 (1 mg) and sodium hydroxide (5 M, 6 μL) was added to the collected solution, followed by a reaction at 90° C. for 5 minutes. The reaction solution was adsorbed onto a tC18 Sep-Pak cartridge, washed with 10 mL of water, and then eluted with 1.5 mL of CH₃CN. The eluted solution was separated in a HPLC system (Waters, Xterra RP-18, 10×50 mm, 10 W) with a 254 nm UV detector and a radioisotope gamma-ray detector. As solvents for the separation, acetonitrile and water were used at a ratio of 45:55 at a flow rate of 3 mL/min under mobile conditions. The [¹⁸F]fluoromethyl group-introduced radiotracer was collected after about 13.5 minutes. To remove the clinically unacceptable HPLC solvents, the collected solution was prepared into a 5% ethanol/saline solution using a tC18 Sep-Pak cartridge.

### Example 2

Hereinafter, a step of preparing 1-(chloromethyl)-3-methyl-4-phenyl-1H-1,2,3-triazol-3-ium triflate, which is an intermediate for the synthesis of a precursor for fluorine-18 labeling, using 1-(chloromethyl)-4-phenyl-1H-1,2,3-triazole as a starting material, is described in detail.

### Step 1: Preparation of 1-(chloromethyl)-3-methyl-4-phenyl-1H-1,2,3-triazol-3-ium triflate

1-(Chloromethyl)-4-phenyl-1H-1,2,3-triazole (387 mg, 2.0 mmol) was dissolved in 4 mL of acetonitrile, and methyl triflate (0.33 mL, 3.0 mmol) was added dropwise thereto at room temperature. The mixture solution was stirred at room temperature for 1 hour, and the reaction solvent was removed, followed by purification by flash column chromatography (MeOH/CH₂Cl₂=5/95), thereby obtaining 710 mg (99%) of the target compound: ¹H NMR (500 MHz, CDCl₃) δ 8.94 (s, 1H), 7.64-7.56 (m, 5H), 6.29 (s, 2H), 4.29 (s, 3H); ¹³C NMR (125 MHz, CDCl₃) δ 144.2, 132.4, 130.0, 129.7, 129.5, 121.5, 120.6 (q, J=318 Hz), 57.2, 39.2. HRMS (FAB) m/z calcd. for [C₁₁H₁₁ClF₃N₃O₃S—OTf]⁺: 208.0642. found: 208.0639.

### Example 3

A step of preparing a precursor for fluorine-18 labeling and a reference material is described in detail below.

### Step 1: Preparation of 1-[2-(N-acetyl-N-4-phenoxypyridin-3-ylaminomethyl)phenoxymethyl]-3-methyl-4-phenyl-1H-1,2,3-triazol-3-ium triflate

Normethyl PBR28 (PBR28-OH, 333 mg, 1.0 mmol) was dissolved in 4 mL of DMF, and t-BuOK (224 mg, 2.0 mmol) and 1-(chloromethyl)-4-phenyl-1H-1,2,3-triazole (360 mg, 1.0 mmol) prepared in Example 1 were added dropwise thereto at 0° C. The reaction mixture was stirred at room temperature for 5 hours, and then water was added thereto to stop the reaction. The reaction mixture was extracted with ethyl acetate, and then purified by flash column chromatography (5% MeOH/CH₂Cl₂), thereby preparing 230 mg (35%) of the precursor for labeling: ¹H NMR (500 MHz, CDCl₃) δ 8.71 (s, 1H), 8.27-8.26 (m, 2H), 7.66-7.56 (m, 5H), 7.41 (t, J=8.0 Hz, 2H), 7.35-7.32 (m, 1H), 7.28-7.25 (m, 2H), 7.15 (d, J=8.0 Hz, 1H), 7.03 (t, J=7.5 Hz, 1H), 6.81 (d, J=8.0 Hz, 2H), 6.56 (d, J=5.5 Hz, 1H), 6.46 (s, 2H), 4.94 (dd, J=84.0 Hz, J=14.5 Hz, 2H), 4.28 (s, 3H), 1.96 (s, 3H); ¹³C NMR (125 MHz, CDCl₃) δ 170.6, 160.7, 153.5, 152.8, 151.2, 151.0, 143.8, 132.1, 131.6, 130.5, 129.9, 129.8, 129.6, 128.8, 128.4, 126.4, 126.3, 124.1, 121.6, 120.5, 113.9, 110.7, 79.7, 46.5, 38.7, 22.2; HRMS (FAB) m/z calcd. for [C₃₁H₂₈F₃N₅O₆S—OTf]⁺: 506.2192. found: 506.2195.

### Step 2: Preparation of N-(2-fluoromethoxybenzyl)-N-(4-phenoxypyridin-3-yl)acetamide

The triazolium triflate precursor (compound 4, 32 mg, 0.05 mmol) was dissolved in 0.5 mL of acetonitrile, and tetrabutylammonium fluoride (20 mg, 0.075 mmol) was added thereto, followed by stirring at 80° C. for 1 hour. The reaction mixture was extracted with methylene chloride, and then purified by flash column chromatography (hexane/EtOAc=50/50), thereby preparing 15 mg (83%) of a reference material (compound 5).

A method of preparing an [¹⁸F]fluoromethyl group-introduced radiotracer from the triazolium triflate precursor prepared in step 1 is described in detail below.

Fluorine-18 produced in a cyclotron was adsorbed onto the cartridge of Chromafix® (PS-HCO₃), followed by elution with methanol/water containing a phase transfer catalyst such as tetrabutylammonium bicarbonate. The eluate was dried by azeotropic distillation, and a solution of the triazolium triflate precursor (2.3 mg) in tert-butanol (0.4 mL) was added thereto. The reaction mixture was heated at 120° C. for 10 minutes, cooled to room temperature, and then dissolved in 10 mL of water. The solution was adsorbed onto a tC18 Sep-Pak cartridge and washed with 10 mL of water, followed by elution with 1.5 mL of CH₃CN. The eluted solution was separated in a HPLC system (Waters, Xterra RP-18, 10×50 mm, 10 μM) with a 254 nm UV detector and a radioisotope gamma-ray detector. As solvents for HPLC, acetonitrile and water were used at a ratio of 45:55 at a flow rate of 3 mL/min under mobile conditions. An [¹⁸F]fluoromethyl group-introduced radiotracer was collected after about 13.5 minutes. To remove the clinically unacceptable HPLC solvents, the collected solution was prepared into a 5% ethanol/saline solution using a tC18 Sep-Pak cartridge.

Meanwhile, in the preparation of the [¹⁸F]fluoromethyl group-introduced radiotracer according to the present invention, a compound substituted with deuterium may be prepared so that the final target compound will be more stably maintained in vivo. Preparation of this compound can be performed in the same manner as the above-described method, except that diiodomethane-deuterium (d2) or triazolium triflate precursor-d2 is used instead of diiodomethane in the two-step labeling method that uses the prosthetic group or in the single-step labeling method that uses the triazolium triflate precursor, thereby preparing an [¹⁸F]fluoromethyl group-introduced radiotracer-d2.

Meanwhile, in order to compare the efficacy of the prepared [¹⁸F]fluoromethyl group-introduced radiotracer as a radiotracer for diagnosis of neuroinflammation, [¹¹C]PBR28 was prepared according to a known method using normethyl-PBR28 as a precursor in an FXC-PRO module (GE Healthcare). The radiochemical yield of preparation of [¹¹C]PBR28 was 20-30%.

Hereinafter, examples for evaluating the usefulness of the [¹⁸F]fluoromethyl group-introduced radiotracer of the present invention for PET imaging of brain neuroinflammation are described in detail below.

### Example 4

### Measurement of In Vitro Binding Affinities of PBR28 and Reference Material for 18-kDa Translocator Protein (TSPO)

Leukocytes were isolated from 50 mL of heparinized whole blood cells by Ficoll-Hypaque gradient centrifugation using a lymphocyte isolation kit, and the isolated leukocytes were freeze-stored. On the day before analysis, the cells were thawed, diluted with the same amount of buffer (50 mM HEPES, pH7.4), and homogenized, followed by centrifugation at 20,000 g at 4° C. for 15 minutes. The obtained leukocytes were re-suspended in 2.4 mL of buffer and stored at −70° C., and the protein concentration was measured using a Bradford assay. To measure in vitro binding affinity, leukocytes (100 μL of re-suspended membrane) were incubated with 100 μL of radioligand ([³H]PK11195 (S.A: 83.4 Ci/mmol), in 1×PBS) and 1 mL of a reaction mixture containing PBR28 or FM-PBR28 (0.124-10,000 nM) and 50 μL of 0.07 nM radioligand ([³H]PK11195) as an inhibition test, at room temperature for 30 minutes. The resulting cells were washed twice using a cell harvester, and then the amount of radioactivity associated with the cells was measured with a beta-counter. In the analysis conditions, the ratio of the specific bound fraction was less than 20% of the total ³H radioactivity. The in vitro binding affinity results were subjected to nonlinear regression analysis using PRISM software in order to calculate the IC₅₀ values of the fluorine-19-substituted reference material and PBR28.

In this case, the reference material showed 8.28±1.79 nM (IC₅₀), and PBR28 showed 8.07±1.40 nM, indicating that it showed a binding affinity similar to that of the reference material.

### Example 5

### Measurement of Lipophilicities of [¹¹C]PBR28 and [¹⁸F]Fluoromethyl Group-Introduced Radiotracer

For the measurement of lipophilicity, each of the [¹⁸F]fluoromethyl group-introduced radiotracer and [¹¹C]PBR28 (about 0.74 MBq) in 5% ethanol/saline was added to and mixed with n-octanol (5 mL) and sodium phosphate buffer (5.0 mL, 0.15 M, pH 7.4), and then lipophilicity was measured four times. The radioactivity of the sample (100 μL) in each sample was measured, and the lipophilicity was calculated as the ratio of counts per minute to sodium phosphate buffer and n-octanol. The lipophilicity of the [¹⁸F]fluoromethyl group-introduced radiotracer was 2.85±0.02, which was similar to that of [¹¹C]PBR28 (3.01±0.01).

### Example 6

### Measurement of In Vitro Stability in Human Serum

For the measurement of the stability of the [¹⁸F]fluoromethyl group-introduced radiotracer, 0.5 mL of 5% EtOH/saline containing the [¹⁸F]fluoromethyl group-introduced radiotracer was mixed with 0.5 mL of human serum, and then the stability of the radiotracer was analyzed by thin-layer chromatography at 37° C. at 0, 10, 30, 60, 120 and 240 minutes. The results of the measurement showed that the [¹⁸F]fluoromethyl group-introduced radiotracer was stable (>98.8%) up to 240 minutes, indicating that the [¹⁸F]fluoromethyl group-introduced radiotracer is stable enough to perform in vivo biological studies.

### Example 7

### PET Imaging in LPS-Induced Brain Neuroinflammation Rat Model

Construction of LPS-Induced Brain Neuroinflammation Rat Model

To construct a neuroinflammation rat model, male Sprague-Dawley rats with a weight of 200-250 g were used. Each of the rats was anesthetized, and the cranium was exposed and then drilled with a bone drill to form a small hole. Thereafter, 50 μg of LPS (lipopolysaccharide) was injected into the rat body by a Hamilton syringe at a flow rate of 0.5 mL/min (AP, 0.8 mm; L, −2.7 mm; and P, −5.0 mm from the bregma). LPS was maintained for 10 minutes to prevent LPS from flowing backward in the Hamilton syringe, and then the small hole of the cranium was filled with wax and the incised scalp was closed.

PET Imaging Protocol

At 4 days after LPS injection into five rats (227.98±3.8 g), positron emission tomography (PET) images were acquired. The rats were subjected to PET imaging for 120 minutes after injection of [¹¹C]PBR28 or the [¹⁸F]fluoromethyl group-introduced radiotracer into the tail vein of the neuroinflammation model. First, a [¹¹C]PBR28 image was acquired from the neuroinflammation model, and after six-half lives (about 3 hours) when the remaining radioactivity disappeared, an image of the [¹⁸F]fluoromethyl group-introduced radiotracer was acquired.

Furthermore, in order to measure the selective/specific binding affinity of the [¹⁸F]fluoromethyl group-introduced radiotracer in the neuroinflammation model, PK11195 (10 mg/kg) or a reference material (5 mg/kg), which specifically binds to TSPO, was injected simultaneously with the [¹⁸F]fluoromethyl group-introduced radiotracer to acquire an inhibition image, and Flumazenil (5 mg/kg) that binds to CBR was injected simultaneously with the [¹⁸F]fluoromethyl group-introduced radiotracer to measure the selective/specific binding affinity of the radiotracer.

In the PET images of [¹⁸F]fluoromethyl group-introduced radiotracer and [¹¹C]PBR28 PET, acquired from the brain neuroinflammation model rats, it was shown that all the two compounds were more selectively accumulated in the LPS-injected inflammatory region than the contralateral region. Furthermore, the uptake of the fluorine-18-labeled radiotracer was at least 3.0 times higher for about 2 hours (p=0.009), and the [¹⁸F]fluoromethyl group-introduced radiotracer showed a faster uptake (4.5 min vs. 20 min) and a high inflammatory region/contralateral region ratio within a shorter time than the [¹¹C]PBR28 image (3.4 times at 30 min vs. 3.4 times at 90 min). In a comparison between time-activity curves (TACs) obtained after injection of each of the [¹⁸F]fluoromethyl group-introduced radiotracer and [¹¹C]PBR28, there was no significant difference in both striata, but the TAC of the [¹⁸F]fluoromethyl group-introduced radiotracer reached a peak earlier after injection than [¹¹C]PBR28 and was lowered slowly. This indicates that, when the [¹⁸F]fluoromethyl group-introduced radiotracer is clinically applied as a radioactive drug, it enables discrimination between a normal brain region and a brain neuroinflammation region within a short time after injection.

Meanwhile, in a selective/specific imaging study, it was shown that the uptake of PK11195 (10 mg/kg) in the inflammatory region was effectively inhibited by about 66% compared to the uptake of the [¹⁸F]fluoromethyl group-introduced radiotracer. Furthermore, the reference material showed a decrease in uptake of 71%. This suggests that the [¹⁸F]fluoromethyl group-introduced radiotracer binds specifically to the brain neuroinflammation factor TSPO. Moreover, the image obtained by injecting the fluorine-18-labeled radiotracer simultaneously with Flumazenil that binds to CBR did not influence the uptake of the fluorine-18-labeled radiotracer in the inflammatory region, indicating that the [¹⁸F]fluoromethyl group-introduced radiotracer selectively binds to peripheral benzodiazephine receptor (=TSPO).

### Example 8

### Measurement of Metabolism of [¹⁸F]Fluoromethyl Group-Introduced Radiotracer in Brain Neuroinflammation Rat Model

The [¹⁸F]fluoromethyl group-introduced radiotracer (about 37 MBq, 5% ethanol/saline) was injected into the vein of the neuroinflammation model rats through the tail vein. After 30 and 60 minutes, the rats were sacrificed, and brain samples were collected therefrom, after which the metabolism of the fluorine-18-labeled radiotracer was measured by HPLC. As a result, the amount of the [¹⁸F]fluoromethyl group-introduced radiotracer in the rat brain was 97.3% 30 minutes after injection and 96.8% 60 minutes after injection. Other radioactive metabolites, excluding 2-3% fluorine-18, were not observed in HPLC up to 60 minutes after injection. In contrast, according to previous studies, it is known that, in the case of [¹¹C]PBR28, radioactive metabolites exist in an amount of about 10-15%, thereby giving false images. This suggests that the [¹⁸F]fluoromethyl group-introduced radiotracer enables more accurate selective/specific imaging of brain neuroinflammation than [¹¹C]PBR28.

Accordingly, based on the above-described results, the [¹⁸F]fluoromethyl group-introduced radiotracer has advantages over [¹¹C]PBR28 in that it can practically diagnose brain neuroinflammation in about 15 patients due to the long half-life of fluorine-18 compared to [¹¹C]PBR28 (109.74 min versus 20.38 min) after it has been produced by a single production process and in that the fluorine-18-labeled radiotracer makes it possible to diagnose brain neuroinflammation even in hospitals in which no cyclotron is provided. Furthermore, the [¹⁸F]fluoromethyl group-introduced radiotracer has advantages in that it shows a high inflammatory region-to-contralateral region ratio within a shorter time after injection than [¹¹C]PBR28, thereby achieving an advantage in that it shortens the time for the diagnosis of patients.

Meanwhile, the fluoromethyl group introduction technology using the single-step fluorine-18 labeling method, which is used in the present invention, has an advantage in that it can substitute an existing carbon-11-labeled radioactive drug with a fluorine-18-labeled radioactive drug while maintaining the biological usefulness of the existing radioactive drug.

The present invention relates to the [¹⁸F]fluoromethyl group-introduced radiotracer for brain neuroinflammation-targeting positron emission tomography, the synthesis thereof, and the method for evaluating biological results using the same. In the present invention, the [¹⁸F]fluoromethyl group-introduced radiotracer is produced either by introducing [¹⁸F]fluoroiodomethane, obtained by labeling the prosthetic group diiodomethane with fluorine-18, into PBR28-OH through a two-step process or by substituting PBR28-OH with fluorine-18 in a high yield through a single-step process using the triazolium triflate precursor. As a result of the comparative evaluation of in vitro binding affinity, lipophilicity and pharmacokinetics in brain neuroinflammation models with those of known [¹¹C]PBR28, it was found that the [¹⁸F]fluoromethyl group-introduced radiotracer exhibited binding affinity and lipophilicity similar to those of [¹¹C]PBR28. Furthermore, in the comparative evaluation of PET images in brain neuroinflammation models, it was found that the [¹⁸F]fluoromethyl group-introduced radiotracer was selectively and specifically taken up in the inflammatory region within a shorter time and was highly stable in the brain neuroinflammation region.

According to the present invention, in the synthesis of the novel [¹⁸F]fluoromethyl group-introduced radiotracer for brain neuroinflammation-targeting PET and the diagnosis of brain neuroinflammation using the fluorine-18-labeled radiotracer, the compound could be desirably labeled with fluorine-18 having a longer half-life than that of [¹¹C]PBR28 while minimizing the structural change of the compound, it was verified that the [¹⁸F]fluoromethyl group-introduced radiotracer gives accurate selective/specific imaging and has pharmacokinetic advantages, and, therefore, it is expected that it can be effectively used as a radiotracer for brain neuroinflammation-targeting PET.

As described above, according to the present invention, the novel [¹⁸F]fluoromethyl group-introduced radiotracer for brain neuroinflammation-targeting PET shows binding affinity and lipophilicity similar to those of the positive control [¹¹C]PBR28, and also shows results in pharmacokinetic evaluation in brain neuroinflammation models, indicating that it can be effectively used for the diagnosis of inflammatory diseases of the central nervous system in place of [¹¹C]PBR28. Furthermore, it can be used for an increased number of patients due to the long half-life of fluorine-18. Moreover, the [¹⁸F]fluoromethyl group-introduced radiotracer makes it possible to diagnose neuroinflammation diseases using positron emission tomography within a shorter time after its injection than [¹¹C]PBR28.

While the present invention has been described in conjunction with the limited embodiments and diagrams, the present invention is not limited to the above-described embodiments, but it will be apparent to those having ordinary knowledge in the art to which the present invention pertains that various modifications and alterations can be made based on the foregoing description.

Therefore, the technical spirit of the present invention should not be defined based on only the above-described embodiments, but should be defined based on the claims as well as equivalents thereto.

What is claimed is:

1. A method for synthesis of an [¹⁸F]fluoromethyl group-introduced radiotracer for brain neuroinflammation-targeting positron emission tomography, wherein a compound obtained by introducing triazolium triflate into normethyl-PBR28 is used as a precursor and a fluoromethyl group is labeled with fluorine-18 in a single step.

2. The method of claim 1, wherein a reference material for the [¹⁸F]fluoromethyl group-introduced radiotracer is N-(2-fluoromethoxybenzyl)-N-(4-phenoxypyridin-3-yl)acetamide which is synthesized either by introducing [¹⁹F]fluoroiodomethane using normethyl-PBR28 as a starting material or by subjecting a triazolium triflate precursor to a substitution reaction with fluorine-19 using tetrabutylammonium fluoride (TBAF), and which is used to identify the [¹⁸F]fluoromethyl group-introduced radiotracer through simultaneous injection of the reference material and the [¹⁸F]fluoromethyl group-introduced radiotracer into HPLC and is also used to evaluate binding affinity of the [¹⁸F]fluoromethyl group-introduced radiotracer for TSPO.

3. The method of claim 1, wherein 1-(chloromethyl)-3-methyl-4-phenyl-1H-1,2,3-triazol-3-ium triflate obtained using 1-(chloromethyl)-4-phenyl-1H-1,2,3-triazole and MeOTf is used as an intermediate for synthesis of the precursor labeled with fluorine-18.

4. A method for evaluating biological results using an [¹⁸F]fluoromethyl group-introduced radiotracer for brain neuroinflammation-targeting positron emission tomography which is synthesized by using a compound, obtained by introducing triazolium triflate into normethyl-PBR28, as a precursor and performing substitution with fluorine-18 in a single step, the method comprising: by using the [¹⁸F]fluoromethyl group-introduced radiotracer, evaluating specificity of the [¹⁸F]fluoromethyl group-introduced radiotracer using PK11195 (8-12 mg/kg) and fluoromethyl-PBR28 (3-7 mg/kg) which are standard materials, and evaluating selectivity of the [¹⁸F]fluoromethyl group-introduced radiotracer using flumazenil (3-7 mg/kg) which binds to central benzodiazepine receptor (CBR).

5. An [¹⁸F]fluoromethyl group-introduced radiotracer for brain neuroinflammation-targeting positron emission tomography, which is synthesized by using a compound, obtained by introducing triazolium triflate into normethyl-PBR28, as a precursor and performing substitution with fluorine-18 in a single step.
